(19) United States
(12) Patent Application Publication
(10) Pub. No.: US 2005/0261174 A1
(43) Pub. Date: Nov. 24, 2005

(54) Insecticidal activity of a cyclic peptide

(76) Inventors: Paul Lewer, Indianapolis, IN (US); Dennis Oscar Duebelbeis, Indianapolis, IN (US); Paul Richard Graupner, Carmel, IN (US); Jim Xinpei Huang, Carmel, IN (US)

Correspondence Address: DOW AGROSCIENCES LLC, 9330 ZIONSVILLE RD, INDIANAPOLIS, IN, 46268, US

(21) Appl. No.: 11/133,935

(22) Filed: May 20, 2005

Related U.S. Application Data
(60) Provisional application No. 60/572,730, filed on May 20, 2004.

(51) Int. Cl. A01N 65/00; C07K 5/12

(52) U.S. Cl. 514/10; 530/317

(57) ABSTRACT

A cyclic peptide isolated from an extract of the bark of a Madagascan plant, having a structure of formula (I), has insecticidal activity.

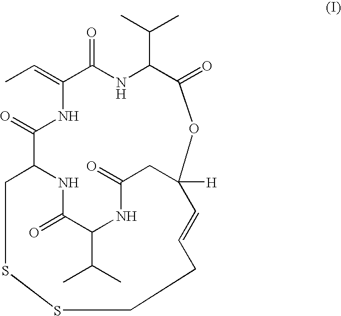

The U.S. Government has a paid-up license in this invention and the right in limited circumstances to require the patent owner to license others on reasonable terms as provided for by the terms of Grant No. 2U01 TW00313-11 awarded by the National Institutes of Health (NIH). This application claims the benefit of U.S. Provisional Application No. 60/572,730, filed on May 20, 2004.

### BACKGROUND OF THE INVENTION

The present invention concerns the insecticidal activity of a cyclic peptide isolated from an extract of the bark of a Madagascan plant. This invention also includes pesticide compositions containing the cyclic peptide and methods of controlling insects using the cyclic peptide.

There is an acute need for new insecticides. Insects are developing resistance to the insecticides in current use. At least 400 species of arthropods are resistant to one or more insecticides. The development of resistance to some of the older insecticides, such as DDT, the carbamates, and the organophosphates, is well known. But resistance has even developed to some of the newer pyrethroid insecticides. Therefore a need exists for new insecticides, and particularly for compounds that have new or atypical modes of action.

### SUMMARY OF THE INVENTION

This invention concerns a natural compound useful for the control of insects. More specifically, the invention concerns the insecticidal activity of the compound of formula (I)

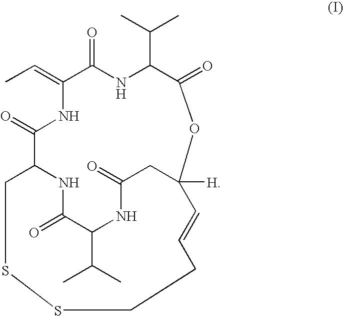

The invention also provides a method of isolating the compound of formula (I) from natural sources as well as new insecticidal compositions and methods of use, which will be described in detail hereinafter.

### BRIEF DESCRIPTION OF THE FIGURES

FIG. 1 is an analytical LC-MS-bioassay chromatogram of the Polyamide Solid Phase Extract (PA-SPE) of crude bark extract MG899.

FIG. 2 is a semi-preparative HPLC chromatogram of the acetonitrile-soluble phase from the PA-SPE eluent of the crude extract. Profile shown is 210 nm UV absorbance resulting from injection of ⅕ (8 mg) of sample. To complete the separation of the whole sample, this process was repeated five times, pooling 16-17 minute region to obtain purified metabolite.

FIG. 3 is the electrospray mass spectra of the active metabolite.
A: Positive ion, low cone voltage (low fragmentation)
B: Positive ion, high cone voltage (high fragmentation)
C: Negative ion, low cone voltage (low fragmentation)
D: Negative ion, high cone voltage (high fragmentation)

FIG. 4 is the 600.13 MHz ¹H NMR spectrum of purified metabolite in MeOH-d₄.

### DETAILED DESCRIPTION OF THE INVENTION

The compound of formula (I) was isolated from an extract of the bark of a Madagascan plant coded MG899 provided under a Madagascar International Cooperative Biodiversity Group Cooperative Research Agreement funded by NIH and administered by Virginia Polytechnic Institute and State University.

Bioassay-guided fractionation led to the isolation of a cyclic peptide of formula (I). The titer of this compound in the bark was estimated to be approximately 13 ppm (milligrams per kilogram), while that in the roots, wood and leaves-plus-fruit was approx. 2, 1 and 0.3 ppm, respectively. Based on NMR and mass spectral data, it was concluded that the cyclic peptide of formula (I) had the same structure as the compound “FR901228” whose structure had been previously reported in the literature; see U.S. Pat. No. 4,977,138; Ueda, H., Nakajima, H., Hori, Y., Fujita, T., Nishimura, M., Goto, T. and Okuhara, M., “FR901228, A Novel Antitumor Bicyclic Depsipeptide Produced by Chromobacterium violaceum No. 968; I: Taxonomy, Fermentation, Isolation, Physico-Chemical and Biological Properties, and Antitumor Activity”, J. Antibiotics, 47 (3), 301, (1994); and Marshall, J. L., Bizvi, N., Kauh, J., Dahut, W., Figuera, M., Kang, M. H., Figg, W. D., Wainer, I., Chaissang, C. L., Megan, Z. and Hawkins, M. J., “A Phase I Trial of Depsipeptide (FR901228) in Patients with Advanced Cancer”, J. Experimental Therapeutics and Oncology, 2(6), 325-332 (2002).

### EXAMPLES

General Analytical and Semi-Preparative HPLC—All analytical and semi-prep LC were performed using a HP 1050 system consisting of autosampler, quaternary pump, vacuum degassing unit and diode array detector (DAD). When needed, a portion (approx. 25%) of the eluent emerging from the diode array detector was routed to an Alltech model 2000 Evaporative Light-Scattering Detector (ELSD) and/or was collected into microtiter plates for biological evaluation using a Gilson FC204 fraction collector. The stationary phase used for this work was hypersil-C8-BDS (250 mm long×4.6 mm ID with 5 μm particle size for analytical work) or HS-hyperprep-C8-BDS (250 mm long×10 mm ID with 8 μm particle size for semi-preparative work).

NMR—All NMR experiments were acquired on a Bruker DRX600 spectrometer operating at 600.13 MHz (proton). The sample was dissolved in 500 μL MeOH-d₄ and any air bubbles were removed with a short burst of ultrasound prior to insertion into the spectrometer. All experiments were acquired using standard Bruker pulse sequences and parameter sets.

Mass Spectrometry—Nominal mass LC/MS was performed using an Agilent 1100 series HPLC with sample output split after DAD (190-700 nm) detection between a Gilson 215 fraction collector (FC) and a MicroMass LCZ mass spectrometer (MS). Split ratio was approximately 9:1, FC:MS. MS full scan (50-1500 amu) data was acquired in low cone voltage (35V) and high cone voltage (85V) for both positive and negative acquisition modes. Sensitive and selective analysis in lower-titer tissue extracts utilized selected ion acquisition at 424 and 541 amu in the low cone voltage positive ion mode. Accurate LC/MS analyses were performed using both the Perceptive Biosystems QSTAR XL and the Micromass QTOF-micro hybrid time-of-flight/quadrupole LC/MS systems. Samples were separated on Hewlett-Packard HP-1100 liquid chromatography (LC) systems with UV diode-array detection. LC separations were performed using Hypersil BDS C-18 column (4.6×250 mm) under linear gradient conditions shown below where: A=10 mM ammonium acetate and B=acetonitrile.

| Time (mins) | Percent A | Percent B |
|---|---|---|
| 0 | 100 | 0 |
| 20 | 0 | 100 |
| 25 | 0 | 100 |
| 27 | 100 | 0 |
| 30 | 100 | 0 |

The post-column effluent stream of each instrument was split approximately 1:4 (ESI:waste), and the ESI portion was introduced into the MS. The instruments were operated in positive electrospray (+ESI) mode using data-dependent triggering between MS and MS/MS modes. Accurate mass analyses on the QTOF-micro were performed using the ‘lock spray’ source, which allowed the sampling of an internal standard (IS) stream at a fixed interval, along with the primary analyte stream. For the IS stream, a solution of Leucine Enkephalin (Sigma L9133, Lot 51K510) at 0.5 ng/mL in 50/50 water/acetonitrile with 0.1% formic acid was introduced at 20 μL/min. Prior to analysis, each instrument was calibrated to within +/−0.005 Da in +ESI using a solution of either spinosyn A or sodium trifluoroacetate.

Beet Armyworm Bioassay Conditions—Lepidopteran diet was dispensed into the wells of a 96-well microtiter plate (100 μL/well) and allowed to cool and solidify. Test samples were dried in the wells of a second 96-well plate then dissolved in acetone-water (50:50; 50 μL) with sonication. The test sample solutions were transferred to the plate containing insect diet and allowed to dry on the surface of the diet. Each well was then infested with 8-10 beet armyworm (BAW) eggs. Test plates were covered with a layer of sterile cotton and then the plate lid. The effects of the test compounds on the development of the insects were evaluated after a 6-7 day incubation period at 28° C. Insecticidal potency was reported as the minimum concentration of compound producing an observable inhibition of larval growth (MIC). This result was expressed either in mass of compound relative to the mass of diet in the whole well (in parts per million, ppm), or as micrograms of compound per square centimeter of diet surface per well.

### Example 1

### Polyamide Solid Phase Extraction (PA-SPE) of Crude Bark Extract

The crude, dark red, plant extract MG899 (7.05 grams) was dissolved in methanol (141 mL, to give 50 mg/mL concentration). Thirty-five “Spe-ed Amide-2” polyamide solid phase extraction cartridges (Applied Separations, Allentown, Pa.; 2 gram/6 mL size) were equilibrated with methanol (8 mL). Four mL of the methanolic bark extract was applied to each cartridge and allowed to pass through under gravity, collecting the eluate. Each cartridge was eluted with a further aliquot of methanol (12 mL), and the eluate was collected and pooled with the initial eluate. The combined methanol eluates were dried under vacuum to give a pale yellow solid (515.6 mg).

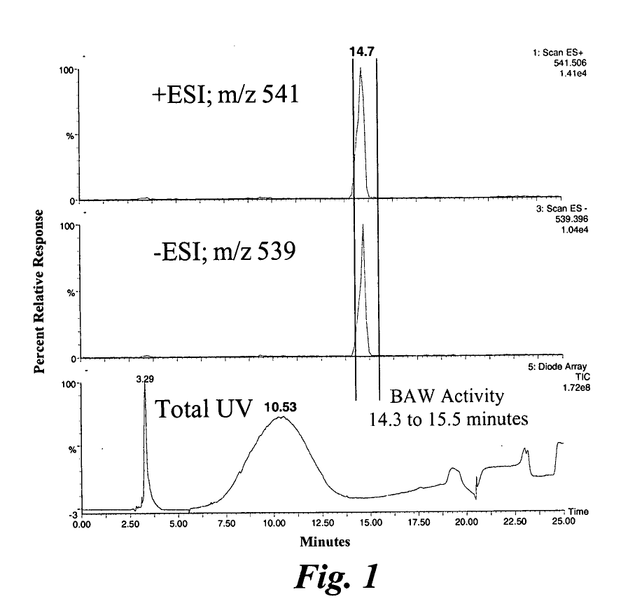

LC-MS analysis of this sample showed a large, broad UV-absorbing peak eluting between 7-13 minutes (FIG. 1) characteristic of tannins, which probably resulted from breakthrough in the PA-SPE step. A BAW-active region detected during LC-MS eluted just after the tannin region, between 14.3-15.5 minutes (FIG. 1). This biologically-active region contained a compound with an accurate molecular mass of 540.2062+/−0.005 Da. The accurate MS/MS spectra of this peak at m/z 540 produced a rich series of fragment ions consistent with a peptide structure.

### Example 2

### Solvent Partitioning of PA-SPE Eluate

The solid from the PA-SPE step was dissolved in EtOAc-water (1:1; 100 mL total). After shaking to partition, the layers were separated by centrifugation and the aqueous phase was extracted with two further aliquots of EtOAc (2×50 mL). The EtOAc and aqueous phases were separately dried under vacuum to give 119.6 and 440.4 mg residue, respectively. Since the EtOAc phase was difficult to solubilize for assay purposes, it was further fractionated by partitioning between acetonitrile and hexane (1:1; 5 mL). After separating the phases by centrifugation, the acetonitrile phase was extracted with a further aliquot of hexane (5 mL), and the acetonitrile and hexane phases were dried under vacuum. This process yielded 45.8 mg and 65.6 mg of acetonitrile-soluble and hexane-soluble material, respectively. BAW run-down bioassay of the acetonitrile, hexane and spent aqueous phases showed that only the acetonitrile phase was insecticidally active.

### Example 3

### Preparative HPLC of Acetonitrile Phase

The acetonitrile-soluble sample from the previous step was dissolved in methanol (500 μL) and chromatographed by repeated injections under semi-preparative HPLC conditions. The column was eluted with a 2-step isocratic solvent system (CG4FC10; Table 1), collecting 0.25-min fractions over 10-25 minutes.

TABLE 1
Elution Conditions for Semi-Preparative HPLC Isolation

| Time (min) | Percent solvent B |
|---|---|
| 0.00 | 35 |
| 25.00 | 35 |
| 25.01 | 40 |
| 35.00 | 40 |
| 35.01 | 35 |
| 45.00 | 35 |

Flow rate = 5 mL/min throughout
Solvent A = 10 mM NH₄OAc
Solvent B = acetonitrile

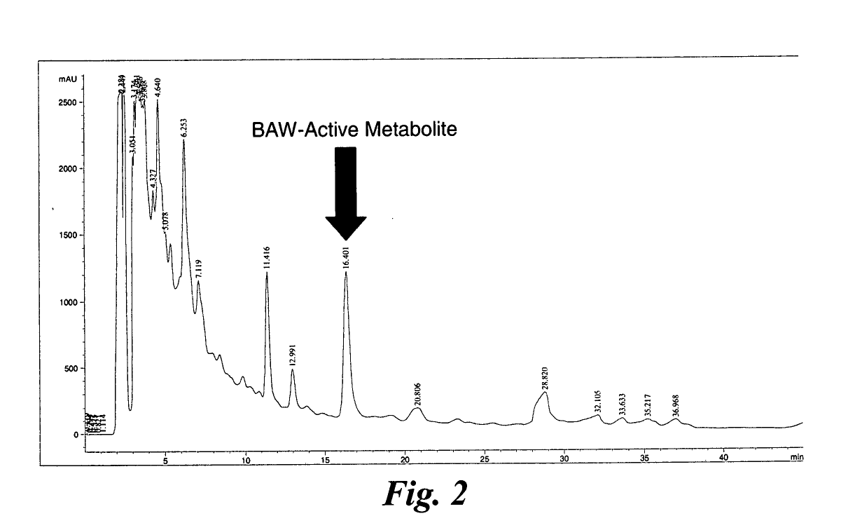

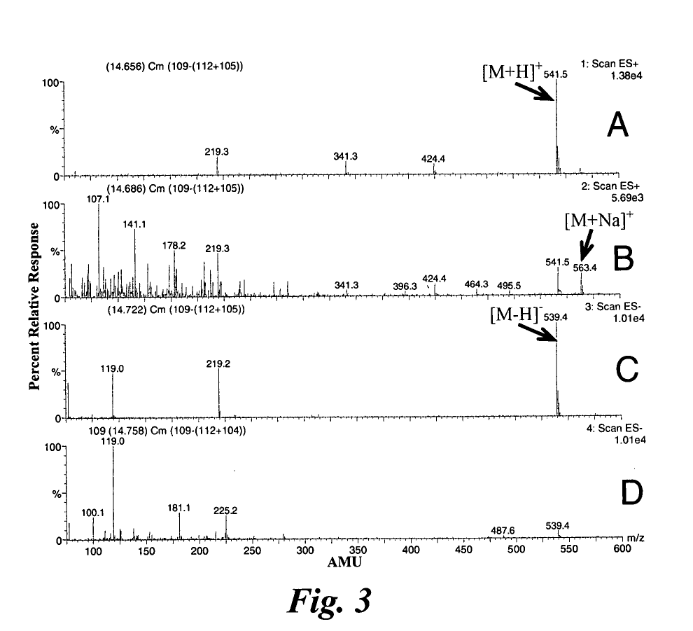

Under these conditions, the compound of interest eluted at approx. 16-17 mins (FIG. 2) and, after drying down, gave 0.9 mg of a white solid of better than 90% purity as determined by HPLC with UV monitoring at 210 nm. The mass spectrum of the purified metabolite matched that from the crude extract (FIG. 3), confirming that the peak originally detected had, indeed, been the true active.

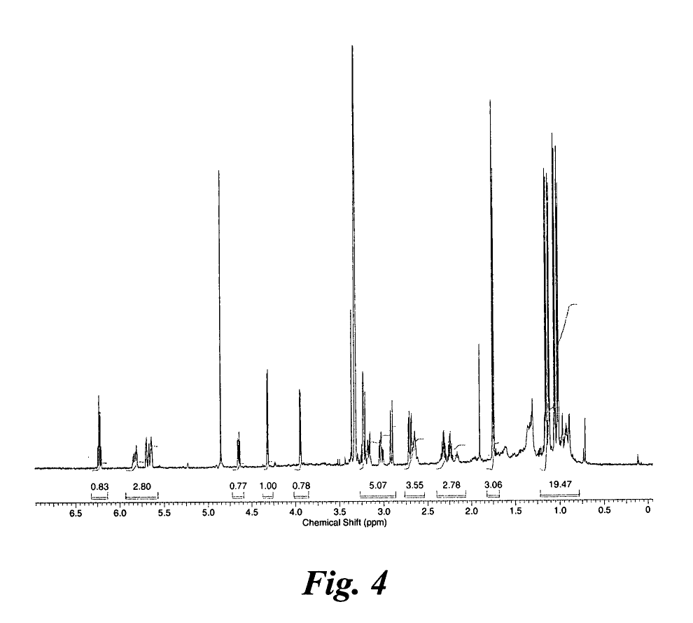

The ¹H NMR spectrum of the purified metabolite was acquired in methanol-d₄, with the large signal due to HOD reduced by presaturation (FIG. 4). The spectrum resembled a peptide, with 5 methyl doublets, and three alpha protons instantly noted. There was sufficient material to acquire a 2D COSY spectrum, allowing the assignment of a number of spin systems. One of the methyl groups resonated at 1.74 ppm, suggesting that it was connected to an sp² carbon atom; the COSY revealed that this was coupled to a proton resonating as a quartet at 6.22 ppm suggesting an allylic methyl group. The other 4 methyl doublets were coupled to multiplets at 2.25 and 2.32 ppm (two methyls to each), with these multiplets each coupled to a doublet at either 3.95 or 4.32 ppm. These systems were assigned as valines. The last remaining alpha proton was a double doublet at 4.64 ppm, and coupled to a slightly diastereotopic methylene at 3.20 and 3.22 ppm—possibly one of a number of different amino acids that consist of this ABX spin system. The remaining signals appeared to make up an extended spin system, consisting of 3 downfield (possibly olefinic) protons and three methylenes at 3.2-2.6 ppm. Searching the literature databases for these spin systems (in particular the 2 valines, the ABX spin system and the allyl group) indicated a small number of potential matches, only one of which had the extended spin system as suggested above (in which one of the downfield methines was revealed as a lactone rather than an olefinic proton). The allyl group in this literature compound reported as “FR901228” was revealed as a dehydrothreonine and the ABX spin system as a cysteine.

FR901228 was, thus, a good NMR match of the purified metabolite in all regards. Further, the accurate mass of the compound, recorded during LC-MS of the crude extract as discussed above, was 540.2062, which agrees well (Δ=1.4 mmu) with that calculated for FR901228 with C₂₄H₃₆N₄O₆S₂=540.2076. Hence, we concluded on the basis of these spectroscopic data that the insecticidal active in MG899 was highly likely to be FR901228.

Insecticide Utility

The compounds of the invention are useful for the control of insects. Therefore, the present invention also is directed to a method for inhibiting an insect which comprises applying to a locus of the insect an insect-inhibiting amount of a compound of formula (I).

The “locus” of insects is a term used herein to refer to the environment in which the insects live or where their eggs are present, including the air surrounding them, the food they eat, or objects which they contact. For example, insects which eat or contact edible or ornamental plants can be controlled by applying the active compound to plant parts such as the seed, seedling, or cutting which is planted, the leaves, stems, fruits, grain, or roots, or to the soil in which the roots are growing. It is contemplated that the compounds might also be useful to protect textiles, paper, stored grain, seeds, domesticated animals, buildings or human beings by applying an active compound to or near such objects. The term “inhibiting an insect” refers to a decrease in the numbers of living insects, or a decrease in the number of viable insect eggs. The extent of reduction accomplished by a compound depends, of course, upon the application rate of the compound, the particular compound used, and the target insect species. At least an inactivating amount should be used. The terms “insect-inactivating amount” are used to describe the amount, which is sufficient to cause a measurable reduction in the treated insect population. Generally an amount in the range from about 1 to about 1000 ppm by weight active compound is used. For example, insects which can be inhibited include, but are not limited to:
Lepidoptera—Heliothis spp., Helicoverpa spp., Spodoptera spp., Mythimna unipuncta, Agrotis ipsilon, Earias spp., Euxoa auxiliaris, Trichoplusia ni, Anticarsia gemmatalis, Rachiplusia nu, Plutella xylostella, Chilo spp., Scirpophaga incertulas, Sesamia inferens, Cnaphalocrocis medinalis, Ostrinia nubilalis, Cydia pomonella, Carposina niponensis, Adoxophyes orana, Archips argyrospilus, Pandemis heparana, Epinotia aporema, Eupoecilia ambiguella, Lobesia botrana, Polychrosis viteana, Pectinophora gossypiella, Pieris rapae, Phyllonorycter spp., Leucoptera malifoliella, Phyllocnisitis citrella
Coleoptera—Diabrotica spp., Leptinotarsa decemlineata, Oulema oryzae, Anthonomus grandis, Lissorhoptrus oryzophilus, Agriotes spp., Melanotus communis, Popilliajaponica, Cyclocephala spp., Tribolium spp.
Homoptera—Aphis spp., Myzus persicae, Rhopalosiphum spp., Dysaphis plantaginea, Toxoptera spp., Macrosiphum euphorbiae, Aulacorthum solani, Sitobion avenae, Metopolophium dirhodum, Schizaphis graminum, Brachycolus noxius, Nephotettix spp., Nilaparvata lugens, Sogatella furcifera, Laodelphax striatellus, Bemisia tabaci, Trialeurodes vaporariorum, Aleurodes proletella, Aleurothrixusfloccosus, Quadraspidiotus perniciosus, Unaspis yanonensis, Ceroplastes rubens, Aonidiella aurantii
Hemiptera—Lygus spp., Eurygaster maura, Nezara viridula, Piezodorus guildingi, Leptocorisa varicornis
Thysanoptera—Frankliniella occidentalis, Thrips spp., Scirtothrips dorsalis
Isoptera—Reticulitermes flavipes, Coptotermes formosanus
Orthoptera—Blattella germanica, Blatta orientalis, Gryllotalpa spp.
Diptera—Liriomyza spp., Musca domestica, Aedes spp., Culex spp., Anopheles spp.
Hymenoptera—Iridomyrmex humilis, Solenopsis spp., Monomorium pharaonis, Atta spp., Pogonomyrmex spp., Camponotus spp.
Siphonaptera—Ctenophalides spp., Pulex irritans
Acarina—Tetranychus spp., Panonychus spp., Eotetranychus carpini, Phyllocoptruta oleivora, Aculus pelekassi, Brevipalpus phoenicis, Boophilus spp., Dermacentor variabilis, Rhipicephalus sanguineus, Amblyomma americanum, Ixodes spp., Notoedres cati, Sarcoptes scabiei, Dermatophagoides spp.
   Insecticidal test for cotton aphid (Aphis gossypii)

To prepare spray solutions, 0.5 mg of test compound was dissolved into 0.5 mL of a 90:10 acetone: water solvent. This 0.5 mL of chemical solution was added to 4.5 mL of water containing 0.025% Tween 20 surfactant to produce a 100 ppm spray solution. Lower concentrations were made by diluting the 100 ppm solution with water containing 0.025% Tween 20.

Squash cotyledons with a single cotyledon leaf were infested with cotton aphid (wingless adult and nymph) 16-20 hours prior to application of spray solution. The solution was sprayed with a hand-held Devilbiss sprayer on both sides of each infested squash cotyledon until runoff. Reference plants (solvent check) were sprayed with 0.025% Tween 20 containing 9% acetone. The plants were allowed to air dry and held for 3 days in a controlled room at 25° C. and 40% RH after which time the test was graded. Grading was by actual count using a dissecting microscope and comparison of test counts to the untreated check. Results are given in Table 2 as percent control based on population reduction versus the untreated.

TABLE 2
Cotton Aphid Test

| Conc | No. of live aphid | | | | | % |
|---|---|---|---|---|---|---|
| PPM | plant 1 | plant 2 | plant 3 | plant 4 | average | control |
| 0 | 107 | 269 | 244 | wilted | 207 | 0.2 |
| 4 | 27 | 40 | 64 | 83 | 27 | 87 |
| 20 | 16 | 4 | wilted | wilted | 10 | 95.2 |
| 100 | 0 | 0 | wilted | wilted | 0 | 100 |

Insecticidal Test for Green Peach Aphid (Myzus persicae).

Cabbage seedlings grown in 3-inch pots, with 2-3 small (3-5 cm) true leaves, were used as test substrate. The seedlings were infested with green peach aphids (wingless adult and nymph) 2-3 days prior to chemical application. Four seedlings were used for each treatment. To prepare spray solutions, 0.5 mg of test compound was dissolved into 0.5 mL of a 90:10 acetone:water solvent. This 0.5 mL of chemical solution was added to 4.5 mL of water containing 0.025% Tween 20 surfactant to produce a 100 ppm spray solution. Lower concentrations were made by diluting the 100 ppm solution with water containing 0.025% Tween 20.

A hand-held Devilbiss sprayer was used for spraying a solution to both sides of cabbage leaves until runoff. Reference plants (solvent check) were sprayed with 0.025% Tween 20 containing 9% acetone. Treated plants were held in a holding room for three days at approximately 25° C. and 40% RH prior to grading. Evaluation was conducted by counting the number of live aphids per plant under a microscope. Results are given in Table 3 as percent control based on population reduction versus the untreated.

TABLE 3
Green Peach Aphid Test

| Conc | No. of live aphid | | | | | % |
|---|---|---|---|---|---|---|
| PPM | plant 1 | plant 2 | plant 3 | plant 4 | average | control |
| 0 | 115 | 104 | 80 | 42 | 85 | 0 |
| 20 | 47 | 53 | 87 | 32 | 55 | 35.8 |
| 100 | 8 | 31 | 30 | 26 | 24 | 72.1 |

Insecticidal Test for Tobacco Budworm (Heliothis virescens), Beet Armyworm (Spodoptera exigua) and Cabbage Looper (Trichoplusia ni) in Dietary Assays.

Dietary assays were conducted in 128-well plastic trays. To prepare a 1000 ppm stock solution, 0.5 mg of test compound was dissolved into 0.5 mL of a 90:10 acetone:water solvent. The test solutions of 125 ppm and lower concentrations were made by sequentially diluting the stock solution with a 90:10 acetone: water solvent. A volume of 50 μl of the test solutions was pipetted upon the surface of 1 mL of lepidopteran diet (Southland Multi-Species Lepidopteran Diet) in each well of 128-well plastic trays. The highest application rate using the 125 ppm solution was equivalent to 3.1 μg/cm². For beet armyworm and cabbage looper, four wells (4 replications) were used for each treatment on each insect species. For tobacco budworm, eight wells (8 replications) were used for each treatment. A second-instar tobacco budworm, two second-instar beet armyworm or two early third-instar cabbage looper larvae were placed upon the treated diet in each well once the solvent had been air-dried. Trays containing the treated diet and larvae were covered with self-adhesive transparent sheets and held in a growth chamber at 25° C., 50-55% RH, and 16 h light: 8 h dark Observation were conducted 1 and 3 days after treatment and infestation. Results are given in Tables 4-6.

TABLE 4
Tobacco Budworm Dietary Test

| | | Symptom | | % Dead | |
|---|---|---|---|---|---|
| PPM | ug/cm2 | 1 day | 3 day | 1 day | 3 day |
| 1 | 0.025 | none | 80% size of | 0 | 0 |
| | | | UTC* | | |
| 5 | 0.13 | 80% size of UTC | 60% size of UTC | 0 | 0 |
| 25 | 0.63 | 60% size of UTC | 40% size of UTC | 0 | 0 |
| 125 | 3.1 | 50% size of UTC, | 20% size of UTC, | 0 | 0 |
| | | 1 paralysis | 1 paralysis | | |

*UTC = solvent-treated control

TABLE 5
Beet Armyworm Dietary Test

| | | Symptom | | % Dead | |
|---|---|---|---|---|---|
| PPM | ug/cm2 | 1 day | 3 day | 1 day | 3 day |
| 1.95 | 0.049 | 90% size of UTC | 30% size of UTC | 0 | 0 |
| 7.8 | 0.195 | 70% size of UTC | 15% size of UTC | 0 | 0 |
| 31.3 | 0.78 | 50% size of UTC | 10% size of UTC | 0 | 0 |
| 125 | 3.1 | 30% size of UTC | all dead | 0 | 100 |

*UTC = solvent-treated control

TABLE 6
Cabbage Looper Dietary Test

| | | Symptom | | % Dead | |
|---|---|---|---|---|---|
| PPM | ug/cm2 | 1 day | 3 day | 1 day | 3 day |
| 1.95 | 0.049 | none | none | 0 | 0 |
| 7.8 | 0.195 | none | 60% size of UTC | 0 | 0 |
| 31.3 | 0.78 | none | 40% size of UTC | 0 | 0 |
| 125 | 3.1 | 2 dead, | 4 dead, | 25 | 50 |
| | | 6 80% size of | 2 partial paralysis, | | |
| | | UTC | 2 40% size of UTC | | |

*UTC = solvent-treated control

Insecticidal Test for Beet Armyworm (Spodoptera exigua) in Topical Assays.

To prepare topical solutions, 0.5 mg of test compound was dissolved into 0.5 ML of a 90:10 acetone:water solvent. A volume of 1 or 2 μl of the test solutions was topically applied to each of early fourth-instar beet armyworm larvae. Six larvae (6 replications) were used for each treatment. This application rate was equivalent to 1 or 2 μg per larva. Treated larvae were individually placed in wells of 6-well plastic plates and fed an artificial diet. Observations were made at one, two and three days after treatment. Results are given in Table 7.

TABLE 7
Beet Armyworm Topical Test

| | | | | % Mortality | | |
|---|---|---|---|---|---|---|
| Dose, | Symptom | | | 1 | 2 | 3 |
| ug/larva | 1 day | 2 day | 3 day | day | day | day |
| 1 | 2 dying w/duiresis, | 1 dead, | 4 dead | 0 | 17 | 67 |
| | 2 unable to right, | 4 dying/duiresis/ | w/duiresis, 1 no | | | |
| | 2 small, 1 OK | no growth, 1 OK | growth, | | | |
| | | | 1 OK | | | |
| 2 | 4 dying | 5 dead, | all dead | 0 | 85 | 100 |
| | w/duiresis/black, | 1 dying/duiresis/ | w/duiresis | | | |
| | 2 small w/little | no growth | | | | |
| | feeding | | | | | |

Compositions

The compounds of this invention are applied in the form of compositions which are important embodiments of the invention, and which comprise a compound of this invention and a phytologically-acceptable inert carrier. The compositions are either concentrated formulations which are dispersed in water for application, or are dust or granular formulations which are applied without further treatment. The compositions are prepared according to procedures and formulae which are conventional in the agricultural chemical art, but which are novel and important because of the presence therein of the compounds of this invention. Some description of the formulation of the compositions will be given, however, to assure that agricultural chemists can readily prepare any desired composition.

The dispersions in which the compounds are applied are most often aqueous suspensions or emulsions prepared from concentrated formulations of the compounds. Such water-soluble, water-suspendable or emulsifiable formulations are either solids, usually known as wettable powders, or liquids usually known as emulsifiable concentrates or aqueous suspensions. Wettable powders, which may be compacted to form water dispersible granules, comprise an intimate mixture of the active compound, an inert carrier, and surfactants. The concentration of the active compound is usually from about 10% to about 90% by weight. The inert carrier is usually chosen from among the attapulgite clays, the montmorillonite clays, the diatomaceous earths, or the purified silicates. Effective surfactants, comprising from about 0.5% to about 10% of the wettable powder, are found among the sulfonated lignins, the condensed naphthalenesulfonates, the naphthalenesulfonates, the alkylbenzenesulfonates, the alkyl sulfates, and nonionic surfactants such as ethylene oxide adducts of alkyl phenols.

Emulsifiable concentrates of the compounds comprise a convenient concentration of a compound, such as from about 50 to about 500 grams per liter of liquid, equivalent to about 10% to about 50%, dissolved in an inert carrier which is either a water miscible solvent or a mixture of water-immiscible organic solvent and emulsifiers. Useful organic solvents include aromatics, especially the xylenes, and the petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are chosen from conventional nonionic surfactants, such as those discussed above.

Aqueous suspensions comprise suspensions of water-insoluble compounds of this invention, dispersed in an aqueous vehicle at a concentration in the range from about 5% to about 50% by weight. Suspensions are prepared by finely grinding the compound, and vigorously mixing it into a vehicle comprised of water and surfactants chosen from the same types discussed above. Inert ingredients, such as inorganic salts and synthetic or natural gums, may also be added, to increase the density and viscosity of the aqueous vehicle. It is often most effective to grind and mix the compound at the same time by preparing the aqueous mixture, and homogenizing it in an implement such as a sand mill, ball mill, or piston-type homogenizer.

The compounds may also be applied as granular compositions, which are particularly useful for applications to the soil. Granular compositions usually contain from about 0.5% to about 10% by weight of the compound, dispersed in an inert carrier which consists entirely or in large part of clay or a similar inexpensive substance. Such compositions are usually prepared by dissolving the compound in a suitable solvent and applying it to a granular carrier which has been pre-formed to the appropriate particle size, in the range of from about 0.5 to 3 mm. Such compositions may also be formulated by making a dough or paste of the carrier and compound and crushing and drying to obtain the desired granular particle size.

Dusts containing the compounds are prepared simply by intimately mixing the compound in powdered form with a suitable dusty agricultural carrier, such as kaolin clay, ground volcanic rock, and the like. Dusts can suitably contain from about 1% to about 10% of the compound.

It is equally practical, when desirable for any reason, to apply the compound in the form of a solution in an appropriate organic solvent, usually a bland petroleum oil, such as the spray oils, which are widely used in agricultural chemistry.

Insecticides and acaricides are generally applied in the form of a dispersion of the active ingredient in a liquid carrier. It is conventional to refer to application rates in terms of the concentration of active ingredient in the carrier. The most widely used carrier is water.

The compounds of the invention can also be applied in the form of an aerosol composition. In such compositions the active compound is dissolved or dispersed in an inert carrier, which is a pressure-generating propellant mixture. The aerosol composition is packaged in a container from which the mixture is dispensed through an atomizing valve. Propellant mixtures comprise either low-boiling halocarbons, which may be mixed with organic solvents, or aqueous suspensions pressurized with inert gases or gaseous hydrocarbons.

The actual amount of compound to be applied to loci of insects and mites is not critical and can readily be determined by those skilled in the art in view of the examples above. In general, concentrations from 10 ppm to 5000 ppm by weight of compound are expected to provide good control. With many of the compounds, concentrations from 100 to 1500 ppm will suffice.

The locus to which a compound is applied can be any locus inhabited by an insect or mite, for example, vegetable crops, fruit and nut trees, grape vines, ornamental plants, domesticated animals, the interior or exterior surfaces of buildings, and the soil around buildings.

Because of the unique ability of insect eggs to resist toxicant action, repeated applications may be desirable to control newly emerged larvae, as is true of other known insecticides and acaricides.

The active compound according to the invention, as such or in its formulations, can also be used in a mixture with known fungicides, bactericides, acaricides, nematicides or insecticides, to widen, for example, the activity spectrum or to prevent the development of resistance. In many cases, this results in synergistic effects, i.e. the activity of the mixture exceeds the activity of the individual components. Examples of particularly advantageous mixing components are the following:

Fungicides:

aldimorph, ampropylfos, ampropylfos potassium, andoprim, anilazine, azaconazole, azoxystrobin, benalaxyl, benodanil, benomyl, benzamacril, benzarnacril-isobutyl, bialaphos, binapacryl, biphenyl, bitertanol, blasticidin-S, bromuconazole, bupite, buthiobate, calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazole, diclofluanid, diclomezine, dicloran, diethofencarb, difenoconazole, dimethirimol, dimethomorph, diniconazole, diniconazole-M, dinocap, diphenylmine, dipyrithione, ditalimfos, dithianon, dodemorph, dodine, drazoxolon, ediphenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, firconazole-cis, furmecyclox, guazatine, hexachlorobenzene, hexaconazole, hymexazole, imazalil, imibenconazole, iminoctadine, iminoctadine albesilate, iminoctadine triacetate, iodocarb, ipconazole, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulphate, copper oxide, oxine-copper and Bordeaux mixture, mancopper, mancozeb, maneb, meferimzone, mepanipyrim, mepronil, metalaxyl, metconazole, methasulphocarb, methfuroxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, pyrazophos, pyrifenox, pyrimethanil, pyroquilon, pyroxyfur, quinconazole, quitozene (PCNB), sulphur and sulphur preparations, tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol triazbutil, triazoxide, trichlamide, tricyclazole, tridamorph, triflumizole, triforine, triticonazole, uniconazole, validamycin A, vinclozolin, viniconazole, zailamide, zineb, ziram and also Dagger G, OK-8705, OK-8801, alpha.-(1,1-dimethylethyl-E-backward.-(2-phenoxyethyl) 1H-1,2,4-triazole-1-ethanol, alpha.-(2,4-dichlorophenyl)-E-backward.-fluoro-b-propyl-1H-1,2,4-triazole-1-ethanol, alpha.-(2,4-dichlorophenyl)-E-backward-methoxy-a-methyl-1H-1,2,4-triazole-1-ethanol, alpha.-(5-methyl-1,3-dioxan-5-yl)-E-backward.-[[4-(trifluoromethyl)phenyl]methylene]-1,2,4-triazole-1,1-ethanol, (5RS,6RS)-6-hydroxy-2,2,7,7-tetramethyl-5-(1H-1,2,4-triazol-1-yl)-3-octanone, (E)-a-methoxyimino)-N-methyl-2-phenoxy-phenylacetamide, isoproyl 1-{2-methyl-1-[[[1-(4-methylphenyl)-ethyl]-amino]-carbonyl]-propyl}-carbamate, 1-(2,4-dichlorophenyl)-2-(1H-1,2,4-triazol-1-yl)-ethanone O -(phenylmethyl)oxime, 1-(2-methyl-1-naphthalenyl)-1H-pyrrol-2,5-dione, 1-(3,5-dichlorophenyl)-3-(2-propenyl)-2,5-pyrrolidinedione, 1-[(diiodomethyl)-sulphonyl]-4-methyl-benzene, 1-[[2-(2,4-dichlorophenyl)-1,3-dioxolan-2-yl]-methyl]-1H-imidazole, 1-[[2-(4-chlorophenyl)-3-phenyloxiranyl]-methyl]-1H-1,2,4-triazole, 1-[1-[2-[(2,4-dichlorophenyl)-methoxy]-phenyl]-ethenyl]-1H-imidazole, 1-methyl-5-nonyl-2-(phenylmethyl)-3-pyrrolidinole, 2′,6′-dibromo-2-methyl-4′-trifluorometoxy-4′-trifluoro-methyl-1,3-thiazole-5-carboxanilide, 2,2-dichloro-N-[1-(4-chlorophenyl)-ethyl]-1-ethyl-3-methyl-cyclopropanecarb-oxamide, 2,6-dichloro-5-(methylthio)-4-pyrimidinyl thiocyanate, 2,6-dichloro-N-(4-tifluoromethylbenzyl)-benzamide, 2,6-dichloro-N-[[4-(trifluoromethyl)-phenyl]-methyl]-benzamide, 2-(2,3,3-triiodo-2-propenyl)-2H-tetrazole, 2-[(1-methylethyl)sulphonyl]-5-(trichloromethyl)-1,3,4-thiadiazol, 2-[[6-deoxy-4-O-(4-O-methyl-.E-backward.-D-glycopyranosyl)-a-D-glucopyranos yl]-amino]4-methoxy-1H-pyrrolo[2,3-d]pyrimidine-5-carbonitrile, 2-aminobutane, 2-bromo-2-(bromomethyl)-pentanedinitrile, 2-chloro-N(2,3-dihydro-1,1,3-trimethyl-1H-indene-4-yl)-3-pyrdinecarboxamide, 2-chloro-N-(2,6-dimethylphenyl)-N-(isothiocyanatomethyl)-acetamide, 2-phenylphenol (OPP), 3,4-dichloro-1-[4-(difluoromethoxy)phenyl]1H-pyrrol-2,5-dione, 3,5-di chloro-N-[cyano-[(1-methyl-2-propynyl)-oxy]-methyl]-benzamide, 3-(1,1-dimethylpropyl-1-oxo-1H-indene-2-carbonitrile, 3-[2-(4-chlorophenyl)-5-ethoxy-3-isoxazolidinyl]-pyridine, 4-chloro-2-cyano-N,N-dimethyl-5-(4-methylphenyl)-1H-imidazole-1-sulphonamide, 4-methyl-tetrazolo[1,5-a]quinazolin-5 (4H)-one, 8-(1,1-dimthylethyl) N-ethyl-N-propyl-1,4-dioxaspiro[4,5]decane-2-methanamine, 8-hydroxyquinoline sulphate, 9H-xanthene-2-[(phenylamino)carbonyl]-9-carboxylic hydrazide, bis-(1-methylethyl)3-methyl-4[(3-methylbenzoyl)-oxy]-2,5-thiophenedicarboxy late, cis-1-(4-chlorophenyl)-2 (1H-1,2,4-triazol-1-yl)-cycloheptanol, cis-4-[3-[4-(1,1-dimethylpropyl)-phenyl-2-methylpropyl]-2,6-dimethyl-morpholine hydrochloride, ethyl[(4-chlorophenyl)-azo]-cyanoacetate, potassium hydrogen carbonate, methanetetrathiol sodium salt, methyl 1-(2,3-dihydro-2,2-dimethyl-1H-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethylphenyl)-DL-alaninate, N-(2,3-dichloro-4-hydroxyphenyl)-1-methyl-cyclohexanecarboxamide, N-(2,6-dimethylphenyl)-2-methoxy-N-(tetrahydro-2-oxo-3-furanyl)-acetamide, N-(2,6-dimethylphenyl)-2-methoxy-N-(tetrahydro-2-oxo-3-thienyl)-acetamide, N-(2-chloro-4-nitrophenyl)-4-methyl-3-nitro-benzenesulphonamide, N-(4-cyclohexylphenyl)-1,4,5,6-tetahydro-2-pyrimidineamine, N-(4-hexylphenyl)-1,4,5,6-tetrahydro-2-pyrimidineamine, N-(5-chloro-2-methylphenyl)-2-methoxy-N-(2-oxo-3-oxazolidinyl)-acetamide, N-(6-methoxy)-3-pyridinyl)-cyclopropanecarboxamide, N-[2,2,2-trichloro-1-[(chloroacetyl)amino]-ethyl]-benzamiide, N-[3-chloro-4,5-bis(2-propinyloxy)-phenyl]-N′-methoxy-methanimidamide, N-formyl-N-hydroxy-DL-alanine-sodium salt, O,O-diethyl[2-dipropylamino)-2-oxoethyl]-ethylphosphoramidothioate, O-methyl S-phenyl phenylpropyl-phosphoraridothioate, S-methyl 1,2,3-benzothiadiazole-7-carbothioate, and spiro[2H]-1-benzopyran-2,1′(3′H)-isobenzofuran]-3′-one;

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations;

Insecticides/acaricide/nematicides:

abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin, Bacillus popilliae, Bacillus sphaericus, Bacillus subtilis, Bacillus thuringiensis, baculoviruses, Beauveria bassiania, Beauveria tenella, bendiocarb, benfuracarb, bensultap, benzoximate, betacyfluthrin, bifenazate, bifentrin, bioethanomethrin, bio-permethrin, BPMC, bromophos A, bufencarb, buprofezin, butathiofos, butocarboxim, butylpyridaben, cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, cis-resmethrin, cispermethrin, clocythrin, cloethocarb, clofentezine, cyanophos, cycloprene, cycloprothrin, cyfluthrin, cyhalothrin, cyhexatin, cypermethrin, cyromazine, deltamethrin, demeton M, demeton S, demeton-5-methyl, diafenthiuron, diazinon, dichlorvos, diflubenzuron, dimethoat, dimethylvinphos, diofenolan, disulfoton, docusat-sodium, dofenapyn, eflusilanate, emamectin, empenthrin, endosulfan, Entomopfthora spp., esfenvalerate, ethiofencarb, ethion, etboprophos, etofenprox, etoxazole, etrimfos, fenamiphos, fenazaquin, fenbutatin oxide, fenitrothion, fenothiocarb, fenoxacrim, fenoxycarb, fenpropatin, fenpyrad, fenpyrithrin, fenpyroximate, fenvalerate, fipronil, fluazinam, flauzuron, flubrocythrinate, flucycloxuron, flucyrnate, flufenoxuron, flutenzine, fluvalinate, fonophos, fosmethilan, fosthiazate, fubfenprox, furathiocarb, granulosis viruses, halofenozide, HCH, heptenophos, hexaflumuron, hexythiazox, hydroprene, imidacloprid, isazofos, isofenphos, isoxathion, ivemectin, nuclear polyhedrosis viruses, lambda-cyhalothrin lufenuron malathion, mecaibam, metaldehyde, methamidophos, Metharhizium anisopliae, Metharhizium flavoviride, methidathion, methiocarb, methomyl, methoxyfenozide, metolcarb, metoxadiazone, mevinphos, milbemectin, monocrotophos, naled, nitenpyram, nithiazine, novaluron, omethoat, oxamyl, oxydemethon M, Paecilomyces fumosoroseus, parthion A, parathion M, permefirin, phenthoat, phorat, phosalone, phosmet, phosphamidon, phoxim, pirimicarb, pirimiphos A, pirimiphos M, profenofos, promecarb, propoxur, prothiofos, prothoat, pymetrozine, pyraclofos, pyresmethrin, pyrethrum, pyridaben, pyridathion, pyrimidifen, pyriproxyfen, quinalphos, ribavirin, salithion, sebufos, silafluofen, spinosad, sulfotep, sulprofos, tau-fluvalinate, tebufenozide, tebufenpyrad, tebupirimiphos, teflubenzuron, tefluthrin, temephos, temivinphos, terbufos, tetrachlorvinphos, theta-cypermethrin, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb, vamidothion, vaniliprole, Verticillium lecanii, YI 5302, zeta-cypermethrin, zolaprofos, (1R-cis)-[5-(phenylmethyl)-3-furanyl]-methyl-3-[(dihydro-2-oxo-3(2H)-furanlidene)-methyl]-2,2-dimethyl cyclopropanecarboxylate, (3-phenoxyphenyl)methyl-2,2,3,3-tetraminethylcyclopropanecarboxylate, 1-[(2-chloro-5-thiazolyl)methyl]tetrahydro-3,5-dimethyl-N-nitro-1,3,5-triazine-2 (1H)-imine, 2-(2-chloro-6-fluorophenyl)-4-[4-(1,1-dimethylethyl)phenyl]4,5-dihydro-oxaz ole, 2-(acetlyoxy)-3-dodecyl-1,4-naphthalenodione, 2-chloro-N-[[[4-(1′-phenylethoxy)phenyl]amino]-carbonyl]-benzamide, 2-chloro-N-[[[4-(2,2-dichloro-1,1-difluoroethoxy)-phenyl]-amino]-carbonyl]-benzamide, 3-methylphenyl propylcarbamate, 4-[4-(4-ethoxyphenyl)-4-methylpentyl]-1-fluoro-2-phenoxy-benzene, 4-chloro-2-(1,1-dimethylethyl)-5-[[2-(2,6-dimethyl-4-phenoxyphenoxy)ethyl]thio]-3 (2H)-pyndazrione, 4-chloro-2-(2-chloro-2-methylpropyl)-5-[(6-iodo-3-pyridihyl)methoxy]-3 (2H)-pyrdazinone, 4-chloro-5-[(6-chloro-3-pyridinyl)methoxy]-2-(3,4-dichlorophenyl)-3-(2H)-pyridazinone, Bacillus thuringiensis strain EG-2348, [2-benzoyl-1-(1,1-dimethylethyl)-hydrzinobenzoic acid, 2,2-dimethyl-3-(2,4-dichlorophenyl)-2-oxo-1-oxaspiro[4.5]dec-3-en-4-yl butanoate, [3-[(6-chloro-3-pyridinyl)methyl]-2-thiazolidinylidene]-cyanamide, dihydro-2-(nitromethylene)-2H-1,3-thiazinie-3 (4H)-carboxaldehyde, ethyl[2-[[1,6-dihydro-6-oxo-1-(phenylmethyl)-4-pyridazinyl]oxy]ethyl]-carba mate, N-(3,4,4-trifluoro-1-oxo-3-butenyl)-glycine, N-(4-chlorophenyl)-3-[4-(difluoromethoxy)phenyl]-4,5-diydro-4-phenyl-1H-pyr azole-1-carboxamide, N-[(2-chloro-5-thiazolyl)methyl]-N′-methyl-N″-nitro-guanidine, N-methyl-N′-(1-methyl-2-propenyl)-1,2-hydrazinedicarbothioamide, N-methyl-N′-2-propenyl-1,2-hydrazinedicarbothioamide, O,O-diethyl[2-(dipropylamino)-2-oxoethyl]-ethylphosphoroamidothioate.

A mixture with other known active compounds, such as herbicides, or with fertilizers and growth regulators is also possible.

1. A composition for controlling insects which comprises a compound of Formula (I) 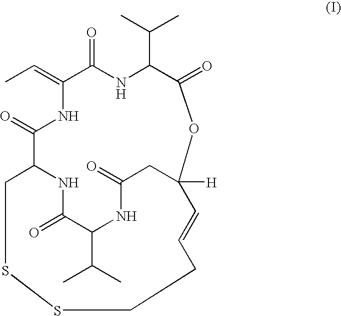
in combination with a phytologically-acceptable carrier.

2. A method of controlling insects which comprises applying to a locus where control is desired a insect-inactivating amount of a compound of Formula (I).
